(19) United States
(12) Patent Application Publication
(10) Pub. No.: US 2023/0310701 A1
(43) Pub. Date: Oct. 5, 2023

(54) DOPA MODIFIED GELATIN FOR WOUND HEALING AND METHODS OF MAKING THE SAME

(71) Applicants: MAYO FOUNDATION FOR MEDICAL EDUCATION AND RESEARCH, Rochester, MN (US); THE REGENTS OF THE UNIVERSITY OF CALIFORNIA, Oakland, CA (US); OBSIDIO, INC., Columbia, SC (US)

(72) Inventors: Alireza Khademhosseini, Los Angeles, CA (US); Hanjun Kim, Los Angeles, CA (US); Yi Chen, Los Angeles, CA (US); Ehsan Jabbarzadeh, Columbia, SC (US); Rahmi Oklu, Chandler, AZ (US)

(73) Assignees: MAYO FOUNDATION FOR MEDICAL EDUCATION AND RESEARCH, ROCHESTER, MN (US); THE REGENTS OF THE UNIVERSITY OF CALIFORNIA, Oakland, CA (US); OBSIDIO, INC., Columbia, SC (US)

(21) Appl. No.: 18/041,973

(22) PCT Filed: Jul. 7, 2021
(86) PCT No.: PCT/US2021/040710

§ 371 Date: Feb. 16, 2023

Related U.S. Application Data
(60) Provisional application No. 63/051,781, filed on Jul. 14, 2020.

(51) Int. Cl. A61L 27/22; A61L 27/54; A61L 27/52; A61L 27/60

(52) U.S. Cl. CPC A61L 27/222; A61L 27/54; A61L 27/52; A61L 27/60; A61L 2300/406; A61L 2300/41; A61L 2300/418; A61L 2400/06

(57) ABSTRACT

A DOPA-Gelatin is disclosed in which the monophenolic group of tyrosine, a prominent amino acid in porcine gelatin, is converted into a catechol group using an enzyme-based DOPA modification technique. The resulting DOPA-Gelatin has been discovered to exhibit good mechanical strength and adhesion. Moreover, in vitro studies show that DOPA-Gelatin has no cytotoxicity with HDF and HaCaT cells, two cells typically involved in the skin wound healing process. Further RT-PCR and angiogenesis investigation showed that DOPA-Gelatin can promote the expression of wound-related genes and facilitate neovascularization. In a full-thickness dorsal defect model in mice, DOPA-Gelatin treated groups decreased the wound closure time and enhanced hair follicle growth. These results demonstrate that the DOPA-Gelatin hydrogel compositions disclosed herein are an effective functional biomaterial that can potentiate the wound healing process.

### CROSS-REFERENCE TO RELATED APPLICATIONS

This application claims the benefit under 35 U.S.C. Section 119(e) of co-pending and commonly-assigned U.S. Provisional Patent Application Ser. No. 63/051,781, filed on Jul. 14, 2020, and entitled “DOPA MODIFIED GELATIN FOR WOUND HEALING AND METHODS OF MAKING THE SAME” which application is incorporated by reference herein.

### STATEMENT REGARDING FEDERALLY SPONSORED RESEARCH AND DEVELOPMENT

This invention was made with government support under HL140951 and HL137193, awarded by the National Institutes of Health. The government has certain rights in the invention.

### TECHNICAL FIELD

The technical field generally relates to 3,4-dihydroxyphenylalanine (DOPA)-modified polymers for biomedical applications. More specifically, the technical field relates to DOPA-gelatin compositions and a one-step process for creating the same using tyrosinase.

### BACKGROUND

The development of novel and unique wound dressings has been pursued extensively over the past decades. As regenerative medicine and tissue engineering have progressed, wound dressings have also evolved into a state-of-the-art technology that promotes wound healing rather than simply covering the wound with gauze^{[1,2]}. Among the variety of wound dressings that have been made, the bioactive/biocompatible hydrogels have gained lots of attention due to their unique advantages such as high water content to hold moisture around the wound area;^{[3-5]} ability to remove necrotic tissue and absorb wound exudate;^{[6,7]} and permeable structures for the diffusion of essential gases such as oxygen, carbon dioxide, and water vapor.^{[8,9]} However, some wound dressing hydrogels also suffer from poor mechanical strength and adhesive properties.^{[10]}

The need to overcome these hurdles and improve the adhesive capability of hydrogels drove researchers to investigate mussels, which show strong adhesion to various surfaces even in wet conditions.^{[11]} Studies have demonstrated that the adhesive abilities of mussels are the result of abundant 3, 4-dihydroxyphenylalanine (DOPA), a special amino acid in protein secreted by the foot organ of mussels.^{[12]} Many natural polymers have been modified with DOPA to improve their adhesion including, but not limited to, DOPA-chitosan^{[13]}, DOPA-PEG^{[14]}, DOPA-HA^{[15]}, and DOPA-alginate^{[16]}. In these studies, DOPA-modified hydrogels not only increase the adhesive property but also improve the bioactivity. For example, catechol-modified hyaluronic acid increases cell viability, reduces apoptosis, and enhances the function of two types of cells (human adipose-derived stem cells and hepatocytes). Dopamine modified alginate hydrogel has better properties for drug adsorption and release.

The processes for making conventional DOPA-containing hydrogels require multi-step preparation and purification methods, which are time-consuming and complicated. Moreover, most DOPA-related hydrogels incorporated the chemical compound dopamine (DA) as the source of the DOPA-structure. Unfortunately, however, the exogenous DA in the synthesis process is usually readily polymerized to polydopamine, which reduces the final material's adhesive properties.^{[17]} Thus, a faster and more direct method of introducing the DOPA structure into polymers is highly desirable. In addition, there is a need in the art for new DOPA-Gelatin hydrogels that can be used in various therapeutic contexts, for example in wound healing.

### SUMMARY

Embodiments of the invention include methods and materials for forming DOPA-Gelatin hydrogels from modified porcine gelatin compositions. Embodiments of the invention further include DOPA-Gelatin adhesive hydrogels made by the methods disclosed herein. Hydrogels formed by the methods disclosed herein have a number of desirable material properties including enhanced in vivo adhesive and in vivo activity profiles. For example, the hydrogels of the invention have been discovered to have an ability to augment biological responses in order to, for example, enhance wound healing.

As discussed below, one way to form DOPA moieties on tyrosine containing polymers such as gelatin without the use of exogenous DA compounds is by using the enzyme tyrosinase. Tyrosinase, well known as polyphenol oxidase, can directly catalyze the phenol groups in tyrosine into catechol groups, the primary chemical group found on DOPA. The one-step synthesis of modified porcine gelatins using tyrosinase as disclosed herein is a more efficient and environmentally friendly approach for making DOPA-modified hydrogels. Only a few hours are needed for synthesis, and the procedure yields material with low toxicity compared to synthesis using chemical compounds. Moreover, compared to other materials, gelatin is readily available and demonstrates favorable biocompatibility. Additionally, it was discovered that the DOPA-Gelatin compositions disclosed herein showed hemostatic ability—an important aspect of the wound healing process.

The invention disclosed herein has a number of embodiments. In one methodological embodiment, tyrosinase was used to catalyze the conversion of tyrosine residues to DOPA with the one-step reaction using porcine gelatin. The in vivo adhesion as well as the efficacy of such DOPA-Gelatin compositions in promoting wound healing were then evaluated (e.g., at the cell and gene expression levels). The results of our studies showed that our one-step method using tyrosinase generates a porcine gelatin hydrogel that contains the catechol groups of DOPA and maintains its adhesiveness and force-bearing properties required of a wound dressing. Our DOPA-Gelatin compositions also improve the proliferation and migration in vitro of both fibroblasts and keratinocytes, which are two important cells involved in the wound healing process. When DOPA-Gelatin was applied to the skin wound area in mice, both the healing rate and hair growth were accelerated as compared to control and untreated gelatin groups.

In one illustrative embodiment of the invention, a method of making 3, 4-dihydroxyphenylalanine (DOPA)-Gelatin includes: (1) providing a solution containing porcine gelatin; and (2) incubating the solution containing porcine gelatin with tyrosinase such that 3, 4-dihydroxyphenylalanine (DOPA)-Gelatin is made. The solution containing porcine gelatin is preferably incubated with tyrosinase for at least one hour (e.g., several hours and preferably about three hours at 37° C.). In one embodiment, the concentration of tyrosinase used is between 100-200 U/mL.

Another embodiment of the invention is a therapeutic composition of matter including porcine gelatin in which substantially all of the tyrosine residues are converted to 3, 4-dihydroxyphenylalanine (DOPA). In some embodiments of the invention, the composition is sterile and comprises a pharmaceutically acceptable carrier. Optionally the composition further comprises at least one additional therapeutic agent such as an antibiotic, an anti-inflammatory agent, a hemostatic agent, an embolic agent, a chemotherapeutic agent or the like. The therapeutic composition of matter can be used in a number of contexts, for example to deliver the composition of matter to a wound site (e.g., skin or non-skin) and promote wound healing.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1A schematically illustrates the DOPA-modification reaction.

FIG. 1B illustrates a photo showing the Tyrosinase concentration dependent modification of DOPA-Gelatin.

FIG. 1C illustrates UV-VIS measurement of enzymatic DOPA-modification.

FIG. 1D illustrates FTIR analysis of DOPA-Gelatin.

FIG. 1E illustrates a graph showing the quantification of DOPA contents by Arnow's method.

FIG. 2A illustrates a graph of lap shear test (left), maximum load (mid) and tensile stress (right) of DOPA-modified gelatin.

FIG. 2B illustrates a graph of burst test (left) and maximum burst pressure (right).

FIG. 2C illustrates rheology test results of DOPA-modified gelatin.

FIG. 3A illustrates representative images of live/dead assay of HDF cells used for showing the in vitro cytotoxicity of DOPA-Gelatin.

FIG. 3B illustrates quantitative analysis of HDF cell viability and proliferation effect of DOPA-Gelatin.

FIG. 3C illustrates representative images of live/dead assay of HaCaT cells.

FIG. 3D illustrates quantitative analysis of HaCaT cell viability and proliferation effect of DOPA-Gelatin.

FIG. 4A illustrates representative images of HDF cell migration assay of DOPA-Gelatin.

FIG. 4B illustrates quantitative analysis of HDF cell migration effect of DOPA-Gelatin.

FIG. 4C illustrates representative images of HaCaT cell migration assay of DOPA-Gelatin.

FIG. 4D illustrates quantitative analysis of HaCaT cell migration effect of DOPA-Gelatin.

FIG. 4E illustrates gene expression analysis result of HDF cell migration sample of DOPA-Gelatin.

FIG. 4F illustrates gene expression analysis result of HaCaT cell migration sample of DOPA-Gelatin.

FIGS. 5A-5D illustrate results of the angiogenesis assay. FIG. 5A shows representative images of angiogenesis for direct method. FIG. 5B is a quantitative analysis of angiogenesis assay for direct method. FIG. 5C shows representative images of angiogenesis for indirect method. FIG. 5D is quantitative analysis of angiogenesis assay for indirect method.

FIGS. 6A-6D illustrate In vivo studies of DOPA-Gelatin. FIG. 6A shows wound healing images on mouse skin model. FIG. 6B shows quantitative analysis of wound healing area. FIG. 6C shows images of histology.

FIG. 7 illustrates DOPA-Gelatin being delivered to a wound site located in skin tissue.

FIG. 8 illustrates a standard curve of absorbance and DOPA concentrations by Arnow's method.

FIG. 9 illustrates representative images of angiogenesis with direct method.

FIG. 10 illustrates quantitative results of branching points and number of tubes for direct method.

FIG. 11 illustrates in vivo degradation test of gelatin and DOPA-Gelatin.

### DETAILED DESCRIPTION OF ILLUSTRATED EMBODIMENTS

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Embodiments of the invention include methods of making 3, 4-dihydroxyphenylalanine (DOPA)-Gelatin. Typically, these methods comprise providing a solution containing porcine gelatin (which is desirable over human gelatin due to its commercial availability); and then incubating the solution containing porcine gelatin with tyrosinase for a specified period of time such as more than 30 minutes, or at least an hour (e.g. at room temp or at 37° C.) etc.; such that 3, 4-dihydroxyphenylalanine (DOPA)-is made. In certain methodological embodiments, the solution containing porcine gelatin is incubated with tyrosinase for at least two or at least three hours at room temperature or 37° C. Typically, the concentration of tyrosinase is not more than 300 U/mL or 200 U/mL, for example, between 100-200 U/mL. Typically, these methods include heating the solution to inactivate the tyrosinase (e.g., at the time that the appropriate 3, 4-dihydroxyphenylalanine (DOPA) is made). In certain embodiments of the invention, the 3, 4-dihydroxyphenylalanine (DOPA)-Gelatin is made in a one-step synthesis reaction.

Embodiments of the invention include a 3, 4-dihydroxyphenylalanine (DOPA)-Gelatin composition made by the methods disclosed herein. Embodiments of the invention include therapeutic compositions of matter comprising porcine gelatin having substantially all (e.g., at least 80%, 85%, 90% or 95%) of the tyrosine residues converted to 3, 4-dihydroxyphenylalanine (DOPA). In certain embodiments of the invention, the composition is substantially free of metallic ions (see, e.g., Y. Chan Choi, J. S. Choi, Y. J. Jung, Y. W. Cho, Journal of Materials Chemistry B 2014, 2, 201, the contents of which are incorporated by reference). In some embodiments of the invention, the composition is sterile and comprises a pharmaceutically acceptable carrier. Optionally the composition further comprises at least one additional therapeutic agent selected from: an antibiotic, an anti-inflammatory agent, a hemostatic agent, an embolic agent, and a chemotherapeutic agent.

Choi et al. used tyrosinase to convert the phenols in tyrosine residues of gelatin extracted from human adipose tissue and quantified the DOPA contents in the formed DOPA-Gelatin.^{[21]} The average tyrosine content in porcine skin gelatin is 26/1000 residues,^{[33]} as compared to 10/1000 residues in human gelatin, and this significant structural difference (e.g. almost three fold greater content of tyrosine residues), makes the material properties of porcine skin gelatin modified according to the methods disclosed herein unpredictable (i.e., as compared to human gelatin). Surprisingly, the methods disclosed herein produced modified porcine gelatin compositions having unexpected and highly desirable material properties. In certain embodiments of the invention, the methods of making these compositions are adapted to form compositions having selected material properties. In some embodiments of the invention, the 3, 4-dihydroxyphenylalanine (DOPA)-Gelatin exhibits a shear strength of at least 2 MPa. In certain embodiments of the invention, the 3, 4-dihydroxyphenylalanine (DOPA)-Gelatin exhibits a burst pressure of at least 6 kPa. In certain embodiments of the invention, the 3, 4-dihydroxyphenylalanine (DOPA)-Gelatin exhibits a load force of at least 60 N. In certain embodiments of the invention, the 3, 4-dihydroxyphenylalanine (DOPA)-Gelatin exhibits a tensile stress of at least 3 MPa. In certain embodiments of the invention, the 3, 4-dihydroxyphenylalanine (DOPA)-Gelatin exhibits a storage modulus of at least 700 Pa. In one illustrative embodiment of the invention, at least 90% of the tyrosine residues of the porcine gelatin have been converted to 3, 4-dihydroxyphenylalanine (DOPA); the composition is sterile and comprises a pharmaceutically acceptable carrier; the 3, 4-dihydroxyphenylalanine (DOPA)-Gelatin exhibits a shear strength of at least 2 MPa; the 3, 4-dihydroxyphenylalanine (DOPA)-Gelatin exhibits a burst pressure of at least 6 kPa; the 3, 4-dihydroxyphenylalanine (DOPA)-Gelatin exhibits a load force of at least 60 N; and the 3, 4-dihydroxyphenylalanine (DOPA)-Gelatin exhibits a tensile stress of at least 3 MPa.

Embodiments of the invention include methods of using the invention, such as a method of using the therapeutic compositions disclosed herein comprising delivering the composition of matter to a wound site. Certain embodiments of the invention include methods of delivering a composition disclosed herein to a preselected site comprising: disposing the composition in a vessel having a first end comprising an opening and a second end; applying a force to the second end of the vessel, wherein the force is sufficient to move the composition out of the vessel through the opening; and then delivering the composition out of the vessel through the opening and to the preselected site, for example, an in vivo site (e.g. at an in vivo location where an individual has experienced trauma or injury such as a skin wound).

Certain embodiments of the compositions of the invention include, for example a pharmaceutical excipient such as one selected from the group consisting of a preservative, a tonicity adjusting agent, a detergent, a viscosity adjusting agent, a sugar and a pH adjusting agent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein. Optionally, the compositions of the invention include one or more therapeutic agents such as an embolic agent, an anti-inflammatory agent, an agent that modulates coagulation, an antibiotic agent, a chemotherapeutic agent, or the like.

Compositions of the invention can be formulated for use as carriers or scaffolds of therapeutic agents such as drugs, cells, proteins, and bioactive molecules (e.g., enzyme). As carriers, such compositions can incorporate the agents and deliver them to a desired site in the body for the treatments of a variety of pathological conditions.

In certain embodiments of the invention, the composition includes a therapeutic agent selected from an anti-inflammatory agent, an embolic agent, and a chemotherapeutic agent. Illustrative embolic agents include, for example, stainless steel coils, absorbable gelatin pledgets and powders, polyvinyl alcohol foams, ethanol, glues, and the like. Illustrative hemostatic agents include, for example, Celox, QuikClot and Hemcon. Certain illustrative materials and methods that can be adapted for use in such embodiments of the invention are found, for example in Hydrogels: Design, Synthesis and Application in Drug Delivery and Regenerative Medicine 1st Edition, Singh, Laverty and Donnelly Eds; and Hydrogels in Biology and Medicine (Polymer Science and Technology) UK ed. Edition by J. Michalek et al. In addition, as scaffolds, compositions of the invention can provide a flexible dwelling space for cells and other agents for use in tissue repair and the regeneration of desired tissues (e.g., for skin, cartilage, bone, retina, brain, and neural tissue repair, vascular regeneration, wound healing and the like).

In some embodiments of the invention, the composition is disposed within a vessel (e.g., a catheter) selected for its ability to facilitate a user modulating one or more rheological properties of the composition (e.g., by applying manual pressure to a 5-FR general catheter or a 2.4-Fr microcatheter). Certain illustrative materials and methods that can be adapted for use in embodiments of the invention are found, for example in Biomedical Hydrogels: Biochemistry, Manufacture and Medical Applications (Woodhead Publishing Series in Biomaterials) 1st Edition; Steve Rimmer (Editor).

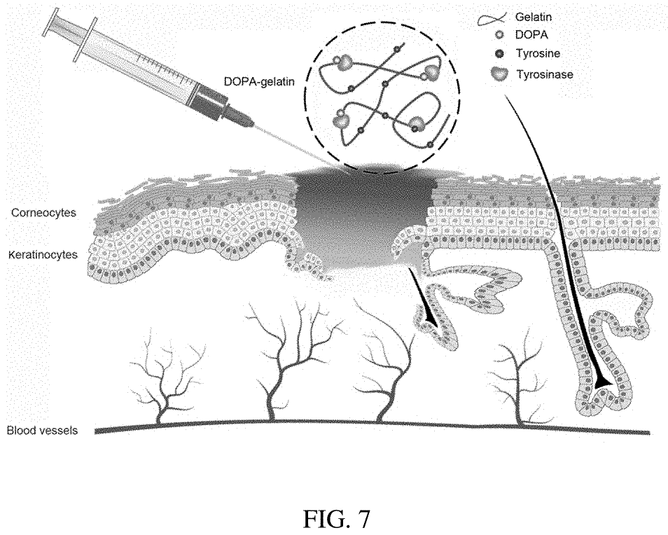

FIG. 7 illustrates the DOPA-Gelatin therapeutic material described herein being delivered to a wound site located in skin tissue. The DOPA-Gelatin therapeutic material was created in a one-step synthesis reaction that involves the enzymatic browning using the enzyme tyrosinase to converts the monophenol group of tyrosine in gelatin into the catechol group of DOPA. The DOPA-Gelatin therapeutic material uses porcine Gelatin as it is readily available commercially. In addition, the porcine gelatin was exposed to tyrosinase for several hours, preferably about three (3) hours to ensure full conversion of all of the tyrosine residues. The DOPA-Gelatin is formed using a 10% (w/w) gelatin solution (type A, G1890, Sigma, CA, USA) that can be prepared by dissolving 1 g gelatin in 10 g Milli-Q water at 80° C. for 1 h. The stock solution of tyrosinase (10 U/μL) which is added to the gelatin solution is made by adding 50 kU tyrosinase powder (T3824, Sigma, MO, USA) into 5 mL Dulbecco's phosphate buffer saline (DPBS, pH6.5, Gibco, CA, USA) at room temperature. Various concentrations of tyrosinase can be used (e.g., 0, 50, 100, 200, 500 U/mL). The reactions can be performed at 37° C. in a mixer such as the Eppendorf ThermoMixer® C (Eppendorf, NY, USA) with an oscillating frequency of 2000 rpm. After the specified incubation time (e.g., around 3 hours), the temperature can be increased to 65° C. for one hour to inactivate the enzyme. The DOPA-Gelatin final solution can be used immediately or stored at −80° C. for later use. Note that the DOPA-Gelatin solution composition is substantially free of metallic ions analogous those used in Choi et al.

It was experimentally determined that optimal conditions exist for the generation of DOPA-Gelatin that has the desired mechanical properties and bioactivity. In one embodiment, the tyrosinase is incubated for about 3 hours to ensure that all or substantially all of the tyrosine residues in the porcine gelatin have been converted to DOPA. In addition, a concentration of tyrosinase between about 100 and about 200 U/mL is desired from a bioactivity perspective. In addition, the use of porcine gelatin is preferred because of the higher prevalence of tyrosine residues as compared to human gelatin. Moreover, porcine gelatin is commercially available in large quantities.

FIG. 7 illustrates DOPA-Gelatin being delivered to a wound location using a delivery device (e.g., syringe). The DOPA-Gelatin can be delivered directly to the site of application on the tissue (e.g., skin tissue). While skin tissue may be healed using the DOPA-Gelatin, other tissue types may also be exposed to the therapeutic DOPA-Gelatin. Moreover, the DOPA-Gelatin may be applied to an external wound but also internal wounds. In addition, in other embodiments, a delivery device such as syringe may not be needed to apply the DOPA-Gelatin. The DOPA-Gelatin may be applied from a container, package, or the like.

Results and Discussion

DOPA-Gelatin Synthesis and Characterization

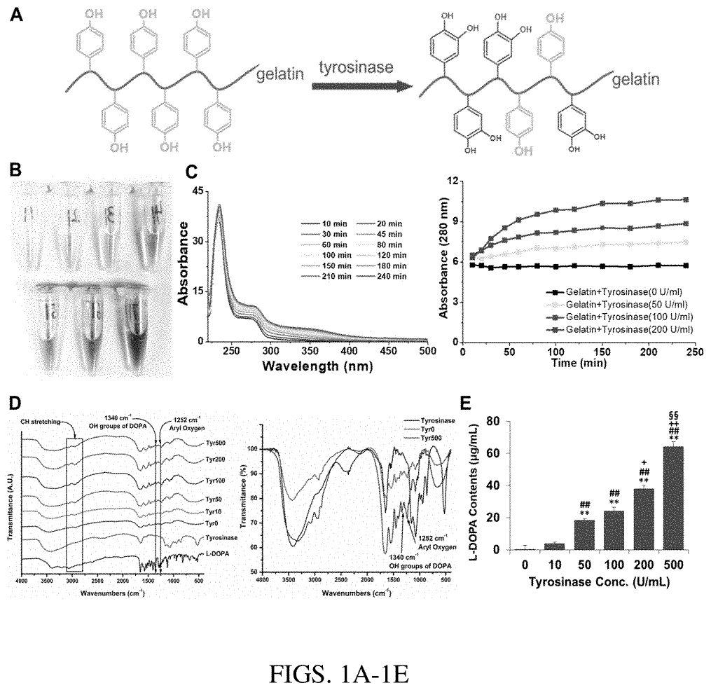

DOPA-Gelatin was generated using a one-step synthesis reaction (FIG. 1A). As one of the polyphenol oxidase (PPO) enzymes involved in the enzymatic browning process, tyrosinase converts the monophenol group of tyrosine in gelatin into the catechol group of DOPA.^{[23-25]} In the experiments disclosed herein, tyrosinase was used in varying concentrations to catalyze the synthesis of porcine-derived DOPA-Gelatin. The gelatin solutions underwent a brown color change (FIG. 1B) after the three-hour reaction, which indicated the formation of the DOPA structure.^{[21]} The brown color deepened with the increasing enzyme concentration. The UV absorption peaks around 280 nm indicate the catechol chemical structure.^{[21, 26, 27]} Thus, the DOPA-Gelatin synthesis reaction was monitored over time. In Choi's study, reaction time was set at 30 min. Here, the effect of reaction time on DOPA formation was investigated. Accompanying extension of the reaction time, the absorption peaks around 280 nm gradually increased, implying the hydroxylation of monophenol in tyrosine (FIG. 1C). The absorbance value did not increase substantially beyond the three-hour reaction, which indicates saturation (FIG. 1C, line chart). Thus, optimized DOPA-Gelatin synthesis time was established at about three hours for converting the monophenol groups to catechol groups. Compared to analogous modification methods used to alter other polymers, the method for altering gelatin is more effective. For example, introduction of DOPA to oxidized alginate requires a multi-step, three-day process consisting of incubation, dialysis, and freeze-drying.^{[28]} DOPA-chitosan hydrogel preparation is also complex and requires stirring, casting into a mold, storage in a refrigerator overnight, and vacuum drying for 24 hours.^{[13]}

To confirm the enzymatic DOPA conversion, Fourier Transform Infrared (FTIR) Spectroscopy was performed. The FTIR absorbance spectrum of pure L-DOPA showed several well-defined bands in accordance with the diverse functional groups present in the structure, namely the amino acid moiety and the hydroxylated benzene ring (FIG. 1D, Table 1). The most important absorption bands to verify the DOPA-Gelatin synthesis are 1340 cm⁻¹ due to OH stretching from the catechol group and 1252 cm⁻¹ due to the oxygen bonded to the aryl ring. For the spectra of DOPA-Gelatin samples, a significant increase in absorption corresponding to the o-diphenol ring was noted in the range of 3670-3115 cm⁻¹ from OH and CN groups (FIG. 1D). Additionally, from 1786-400 cm⁻¹, an increase in absorption bands similar to those of pure L-DOPA were observed.^{[29,30]} These results provide strong evidence that the hydroxylation of monophenol residues in tyrosine had occurred satisfactorily.

TABLE 1

| Frequency (cm⁻¹) | Functional group |
|---|---|
| 3600-2400 | Broad band for O—H/N—H |
| | stretching and H-bonding |
| 3070 | N—H stretching |
| 3210 | C—N stretching |
| 2800-2100 | Vibrations of aryl or aliphatic C—H bonds |
| 1340 | O—H stretching from aryl ring |
| 1252 | Aryl oxygen |

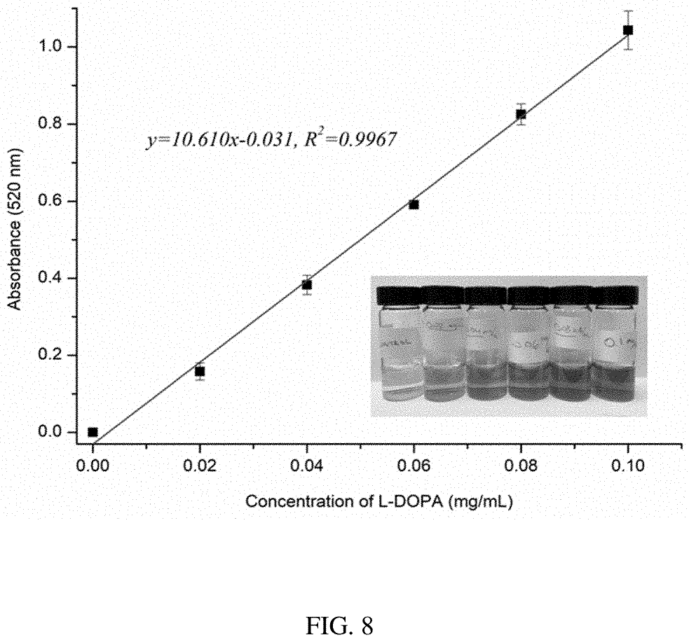

To evaluate the DOPA modification, an Arnow assay was utilized as it provides a useful colorimetric indication of the chemical groups present in a compound.^{[31]} Specifically, it could detect the amount of DOPA in the presence of tyrosine without interference.^{[32]} Same with Arnow's protocol, the o-diphenol group in the DOPA structure reacts with nitrite to produce a bright red chromophore in alkaline solutions (FIG. 8). Absorbance at 520 nm was monitored to quantify the presence of DOPA. The absorbance values and DOPA concentrations appeared to be fairly linearly related, indicating a positive correlation between the variables. DOPA content for each sample was calculated by the standard curve (FIG. 1E). The average DOPA concentration in the Tyr100 sample was found to be 24.12±2.4011 g/mL, much higher than values reported in a previous study.^{[21]} Because the average tyrosine content in porcine skin gelatin was 26/1000 residues,^{[33]} and 10/1000 residues in human gelatin, a reaction time of three hours was used to allow the conversion of monophenol group to DOPA to approach saturation. In summary, a one-step method for the generation of DOPA-Gelatin was created that is compatible with biomedical studies; this method is simpler and less toxic than comparable methods for gelatin modification and DOPA conversion on other biopolymers.

Evaluation of the Adhesive Properties of DOPA-Modified Gelatin

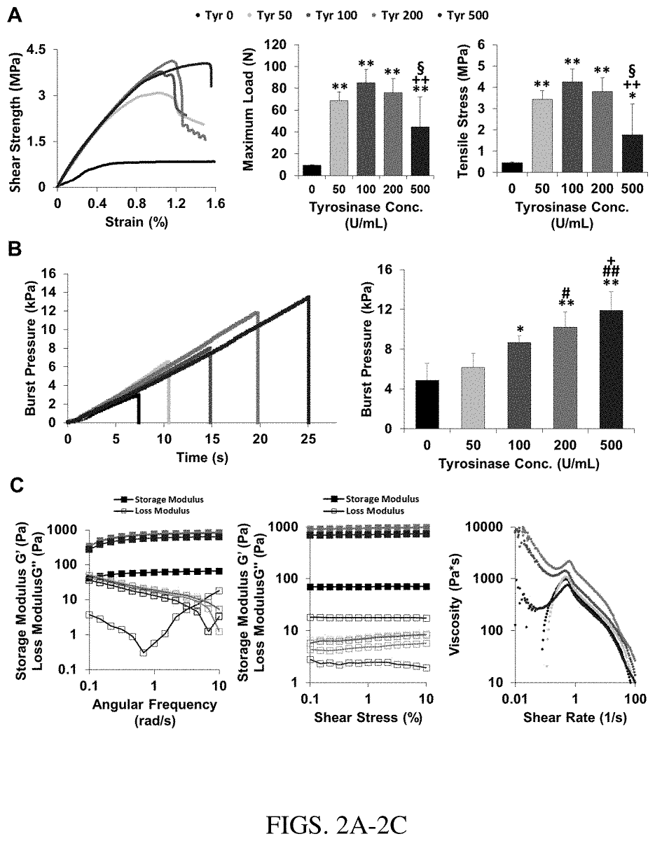

Several different methods were employed to evaluate the adhesive properties of DOPA-Gelatin. The lap shear test is the most commonly used experimental procedure to characterize adhesive behaviors due to its simplicity.^{[34,35}] The test piece is subjected to shear stress by applying a tensile load axially to the two lapped substrates. The point of maximum load and the start of failure (first drop in load) are shown in FIG. 2A. Compared to pure gelatin, all DOPA-Gelatin showed significant increases in the shear strength. The tensile strength of pure gelatin was 0.47±0.03 MPa, while other DOPA-Gelatin samples reached much higher. Specifically, among different DOPA-Gelatins, the Tyr100 sample showed the highest load force of 84.9±12.4 N and tensile stress of 4.25±0.62 MPa. It indicated that the DOPA structure could increase the adhesive property of gelatin by improving its strength and resistance to tension. Interestingly, with the increase of enzyme concentration, the maximum load force and tensile stress decreased. During the enzymatic reaction, tyrosinase was not only involved in the hydroxylation of monophenol but also the conversion of an o-diphenol to the o-quinone.^{[36]} The oxidation reaction of DOPA to DOPA-quinone subsequently led to the formation of covalent bonds that contributed to the cohesion of the adhesives.^{[37,38]} The unoxidized catechol form of DOPA is primarily responsible for adhesion, and catechol oxidation is detrimental to its adhesive ability since the formed o-quinones are non-adhesive.^{[39, 40]} The DOPA-quinone could be monitored by UV-Vis at a peak of approximately 380 nm.^{[27]} With the extension of the reaction time, absorbance around 380 nm also increased (FIG. 1C), which indicated DOPA-quinone formed with greater exposure to tyrosinase. For Tyr500 samples, due to the higher tyrosinase concentration, more DOPA-quinone formed, which deteriorated the adhesion of DOPA-Gelatin. Production with tyrosinase at concentrations of 100-200 U/mL was undertaken to maximize the adhesive properties of the material.

The burst pressure test is another method to investigate the capacity of hydrogels to withstand pressure and adhere to tissue to seal and prevent leakage.^{[41]} A material's burst pressure can be affected by two properties: cohesive (forces within the material to withstand pressure) and adhesive (attachment to the surface) properties while the former has a greater contribution.^{[42]} Moreover, the burst pressure also increased with DOPA content. For the Tyr500 sample, although it had lower adhesion than Tyr100 and Tyr200, it showed the greatest burst pressure of 11.9 kPa due to its strong cohesion. Too much cohesion may result in stiff materials without significant affinity for a surface, thus lowering the adhesion.^{[43]} Without crosslinking, the burst pressure of DOPA-Gelatin had similar performance compared to some commercially available surgical sealants.^{[44]} Based on these results, further experiments were performed with the highest adhesive properties yielded by the tyrosinase concentrations of 100-200 U/mL, using much less enzyme than reported in earlier studies.^{[21]}

Rheology is also commonly used to characterize hydrogel mechanical properties as it is fast, sensitive, and requires a small sample quantity.^{[45]} It is a powerful tool to characterize the viscoelasticity of hydrogels.^{[46]} To further study the mechanical properties of DOPA-Gelatin, rheology tests were employed. To be considered a hydrogel, a material must meet some requirements according to its rheological behavior: storage modulus (G′) must be relatively independent of the frequency of deformation and G′ must be higher than the loss modulus (G″).^{[47]} An amplitude sweep of shear strain from 0.1% to 10% and frequency sweep from 0.1 rad/s to 10 rad/s validated that all samples presented hydrogel-like behavior (FIG. 2C). DOPA-Gelatin samples showed significantly higher storage modulus than pure gelatin. Although the differences among DOPA-Gelatin groups were not obvious, storage modulus of Tyr100 and Tyr200 were 964 Pa and 942 Pa respectively, relatively higher than 718 Pa for Tyr500. The results of the rheological tests revealed that DOPA-Gelatin formed an elastic network with desirable mechanical properties. All samples exhibited declining viscosity on logarithmic shear rate scales from 1 to 100 s⁻¹, which indicates that the material also has shear-thinning properties.

Cytotoxicity and Cytocompatibility of DOPA-Gelatin In Vitro

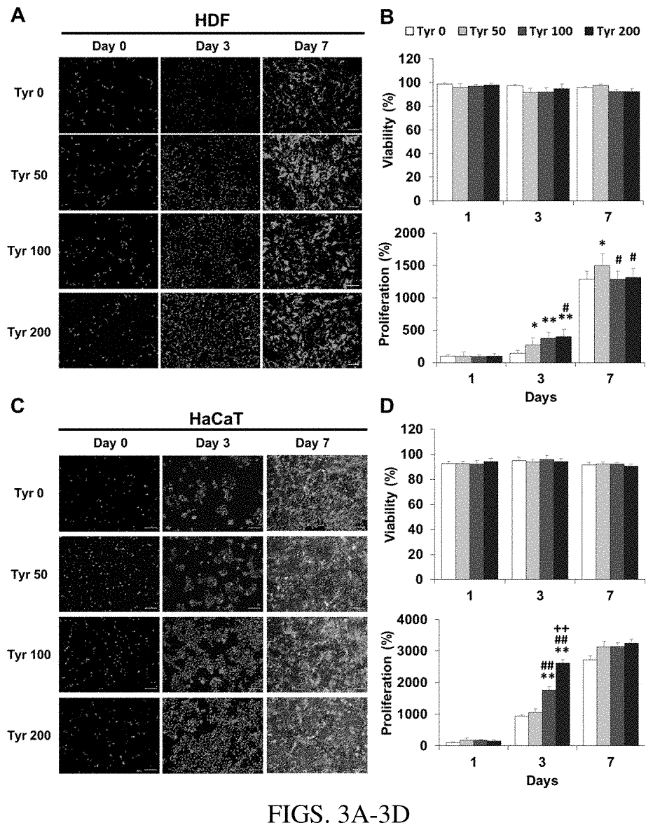

To access the cytotoxicity of DOPA-Gelatin, human dermal fibroblasts (HDF) and human keratinocyte (HaCaT) cells were used as they are closely associated with skin wound healing.^{[48]} Both of them respond to the inflammatory phase in the cutaneous repair/regeneration process. Inflammatory signals activate their proliferation and maturation, which are essential for wound healing.^{[49]} Fibroblasts are shown to deposit matrix-related proteins such as collagen, which make up the basement membrane that separates the epidermis and dermis, while keratinocytes form tight junctions forming a barrier to pathogens and produce hair follicles.^{[50,51]} The results showed that HDF cells grew well on different DOPA-Gelatin samples (FIG. 3A). All four concentrations of DOPA-Gelatin showed no cytotoxicity as cells proliferated for up to 7 days. On Day 7, HDF cell densities on DOPA-Gelatin samples were significantly higher than on pure gelatin, and cells seeded on DOPA-Gelatin had a rich cytoplasm with a more clustered morphology. Cell viability on both gelatin and DOPA-Gelatin samples remained greater than 90% and did not show a statistically significant difference between groups, which indicates that there was no cytotoxicity caused by DOPA-Gelatin. The proliferation rate of cells on DOPA-Gelatin after 7 days was approximately 13 times that of the original cell concentration, which was consistent with cells seeded on pure gelatin (FIG. 3B). The results support the conclusion that the conversion of phenol to catechol groups on tyrosine leads to greater fibroblast growth, which could play a role in the recovery of the dermis.^{[52,53]}

Moreover, both gelatin and DOPA-Gelatin samples showed beneficial effects on HaCaT cells, which can be found in the epidermis of the skin (FIG. 3C). However, by Day 3, differences between images indicated that the cell density was dependent on the tyrosinase concentration. Higher tyrosinase concentrations led to materials that achieved greater HaCaT cell density. As shown in FIG. 3D, the proliferation rate after three days increased from 930% to 2612% as tyrosinase concentration rose from 0 to 200 U/mL. After 7 days, cell viabilities for DOPA-Gelatin samples were all above 90%, and the proliferation rate reached more than 3000%, much higher than that of HDF cells.

According to the experiments conducted herein, DOPA-Gelatin has high cytocompatibility and assists both HaCaT and HDF cells in wound healing. Although a typical sealant may block exposure of the injury site to outside pathogens while cells slowly migrate and proliferate, DOPA-Gelatin provides bioactive cues that clearly increase the proliferation of two different cell types and speed up the healing of the two skin constituents.

The Effect of DOPA-Gelatin on Cell Migration In Vitro

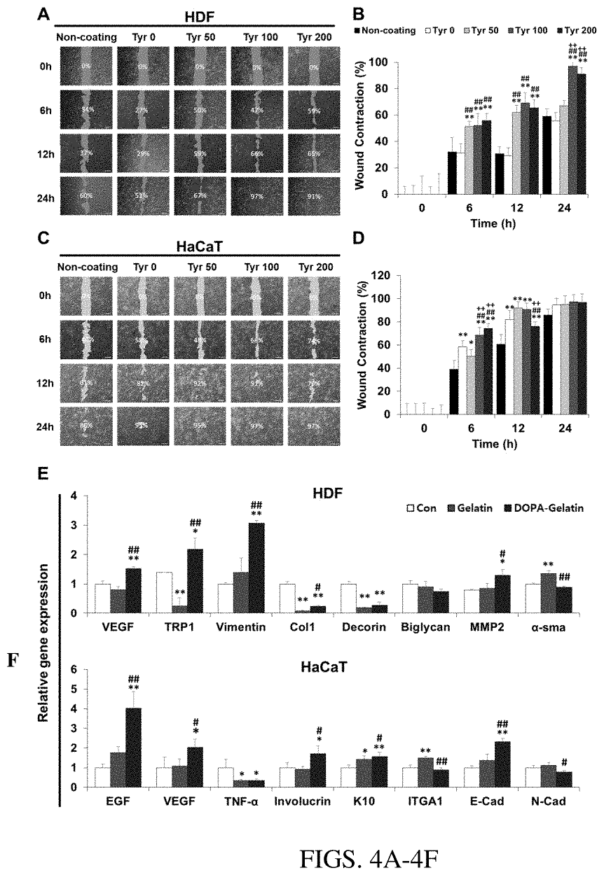

To evaluate cell migration, a scratch wound assay was implemented. This assay involves the creation of a gap in a confluent cell monolayer to mimic a wound and monitor the subsequent cell motion. HDF cells showed faster migration on the DOPA-Gelatin samples than on non-coated or pure gelatin-coated plates (FIG. 4A). At 24 h, wound contraction rates on Tyr100 and Tyr200 samples were almost identical, reaching above 90% which was significantly higher than other groups with rates around 60%. Most notably, Tyr100 facilitated a wound closure rate of 97% (FIG. 4B). It indicated that DOPA-Gelatin could facilitate HDF migration and promote the deposition of extracellular matrix (ECM) components to form the dermal layer. HaCaT cells showed similar migration behavior (FIGS. 4C, 4D). Tyr100 and Tyr200 samples had the greatest migration rate at 6 h, while other groups were relatively low. But after 24 h, unlike HDF, the wound contraction rates between each group did not show a difference, all above 95%. According to these results, both keratinocytes and fibroblasts migrated well on DOPA-Gelatin compared to unmodified gelatin and control groups, especially for Tyr100 and Tyr200 samples.

Cell migration is essential to wound healing and tissue remodeling. In the course of wound healing, keratinocytes migrate from the basal population around the wound edge to cover the lesion and restore the barrier function of the skin.^{[55]} The dermal fibroblasts may also migrate into the wound site, where they synthesize the provisional ECM required for skin wound contraction.^{[56,57]} It was demonstrated that DOPA-Gelatin is a good candidate for epidermal and dermal layer healing and its potential healing effects in the in vivo wound closure experiments.

Quantitative Real-Time Polymerase Chain Reaction (RT-PCR) Assay

Aside from cell migration, gene expression is constantly changing to coordinate a repair response to prevent lasting damage to the wound site. Wound healing includes various processes, interactions between cells and their surrounding microenvironments, which include cellular migration, proliferation, differentiation, angiogenesis, epithelialization, matrix deposition, and remodeling.^{[58]} Therefore, the gene expression that contributed to these processes was analyzed. The results showed both vascular endothelial growth factor (VEGF) and epidermal growth factor (EGF) had the highest expression level in the DOPA-Gelatin group. HDF showed greater expression of MMP2 on DOPA-Gelatin compared to other groups (FIG. 4E). It is well known that VEGF stimulates wound healing through multiple mechanisms including collagen deposition, angiogenesis, and epithelialization.^{[59]} EGF, another well characterized growth factor, is synthesized by keratinocytes which can stimulate the re-epithelialization and increase the tensile strength of skin incisions.^{[60,61]} In addition, vimentin directly coordinates fibroblast proliferation, collagen accumulation, keratinocyte transdifferentiation, and re-epithelialization in wound healing. It is an intermediate filament involved in cell anchorage as well as the epithelial-to-mesenchymal migration process. Loss of vimentin is also known to contribute to a severe deficiency in fibroblast growth.^{[62]} Matrix metalloproteinase 2 (MMP2) was reported to be involved in the remodeling of the stroma and reformation of the basement membrane.^{[63, 64]} During the anagen phase of hair growth, mature melanocytes synthesize melanin from tyrosine; this is controlled by tyrosinase-related protein 1 (TRP1).^{[65]} The results showed that the DOPA-Gelatin group had the greatest expression of TRP1 and that it may facilitate hair growth during wound healing. In summary, DOPA-Gelatin was proved to promote many key processes of wound healing and showed its potential for biological activation.

Angiogenesis Assay

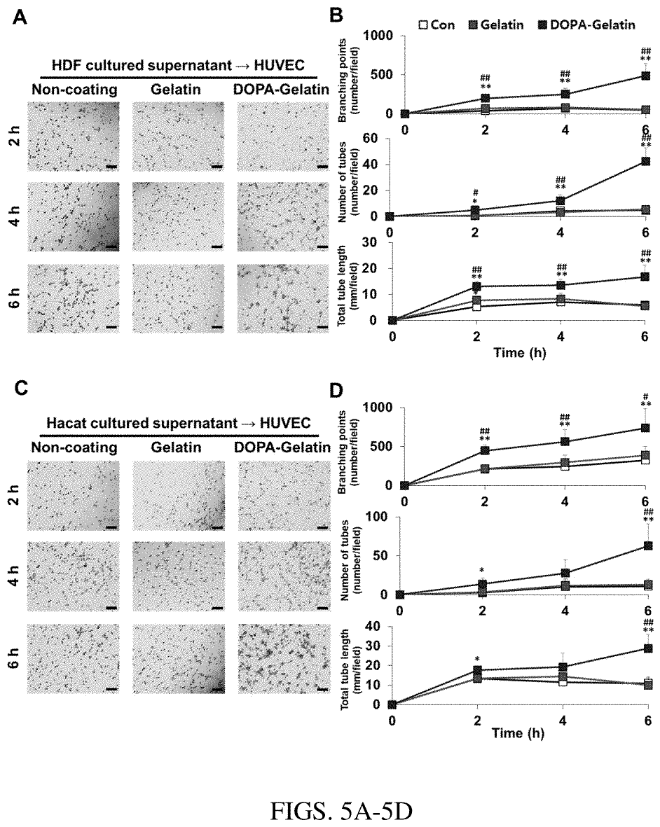

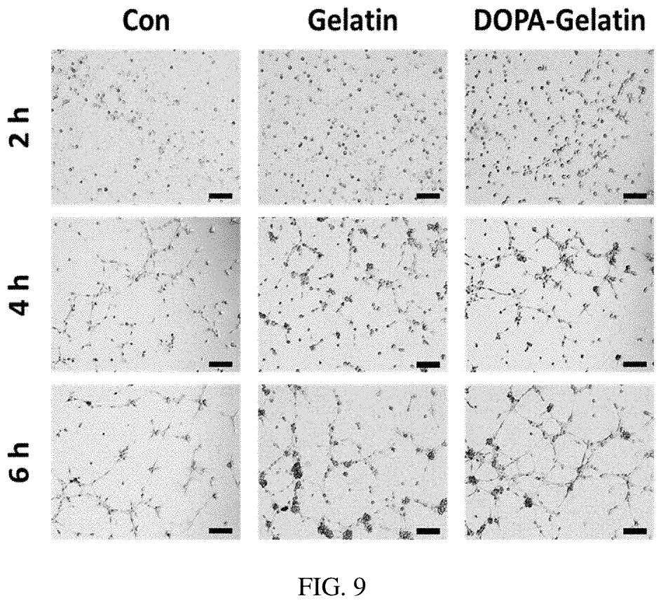

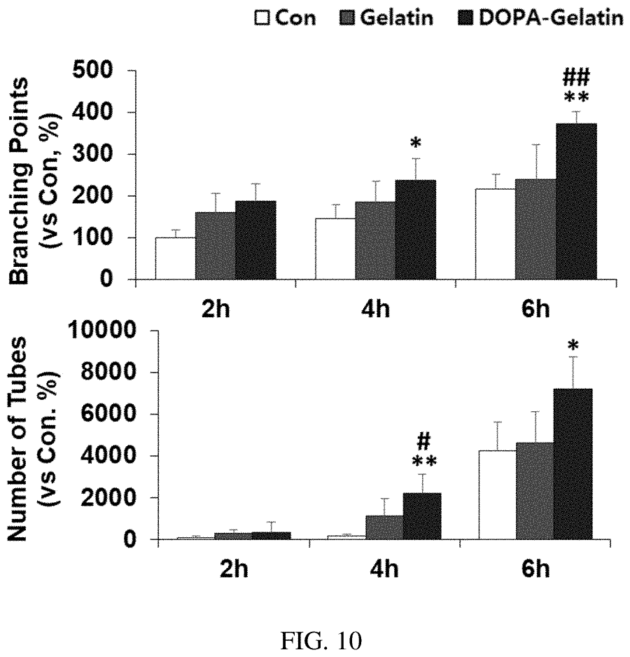

Based on gene expression analysis, the effect of DOPA-Gelatin on vascularization was also investigated. By adding gelatin or DOPA-Gelatin samples to dishes of human umbilical vein endothelial cells (HUVECs), the effect of DOPA-Gelatin on tube formation was directly monitored. In the HUVEC growth medium, vascular tube formation was observed as early as 4 hours after seeding and was confirmed at 6 hours (FIG. 9). Specifically, tube formation in the DOPA-Gelatin group appeared 2 hours earlier than the control and gelatin groups. In addition, quantification showed that the DOPA-Gelatin group had higher results in both branching points and the number of tubes (FIG. 10). At 6 h, the number of tubes in the DOPA-Gelatin group was around 1.7 times that of the other two groups. These results indicate that DOPA-modification influences the bioactive properties of vascular endothelial cells, helping with the re-endothelialization of blood vessels, thus promoting wound healing.^{[68]} Alternatively, the indirect method includes the application of conditioned media collected from dishes of HDF and HaCaT cells to culture HUVECs. Using this method, similar trends were observed for both HDF and HaCaT groups. At the 6 h time point, DOPA-Gelatin showed significantly better results for each parameter compared to the control and gelatin groups (FIG. 5). While the differences between control and gelatin groups were not drastic, they corresponded with VEGF expression in these two cell incubation systems. As shown in FIG. 4E, both HDF and HaCaT cells showed the greatest relative expression of VEGF in DOPA-Gelatin groups while the gelatin group was close with that seen in the control groups. It proved that augmented secretion of VEGF induced by DOPA facilitated angiogenesis.^{[69]} Compared with the control group, VEGF expression was greater in HaCaT than in HDF. From the results shown in FIGS. 5B and 5D, it can be seen that at 6 h, the number of tubes in the DOPA-Gelatin group cultivated with HaCaT cultured supernatant was larger.

The remodeling and establishment of new blood vessels is one of the critical factors in wound healing as vessels supply cells at the wound site with nutrition and oxygen; these angiogenic activities proceed concurrently during all phases of the reparative process.^{[66,67]} Both tube formation images and quantification results here demonstrate the superiority of DOPA-Gelatin in the angiogenesis process. The material can accelerate vessel formation and shorten the wound healing period overall.

In Vivo Studies

In Vivo Degradation Test

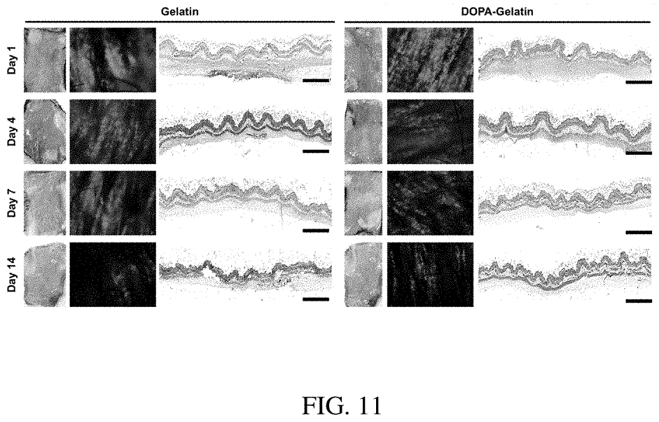

To evaluate the degradation in vivo, pure gelatin and DOPA-Gelatin were implanted in mice, both labeled with fluorescein isothiocyanate (FITC), into the dorsum subcutis and monitored them over 14 days. As shown in the fluorescent images in FIG. 11, both gelatin and DOPA-Gelatin remained subcutaneous for 14 days. However, as time passed, the fluorescent intensity decreased due to degradation. On day 14, hematoxylin and eosin (H&E) staining showed that both gelatin and DOPA-Gelatin implantation caused minor inflammation during degradation; however, there was no indication of infection, specific cell infiltration (neutrophils and lymphocytes), or progression to chronic inflammation.

In Vivo Skin Wound Healing Study

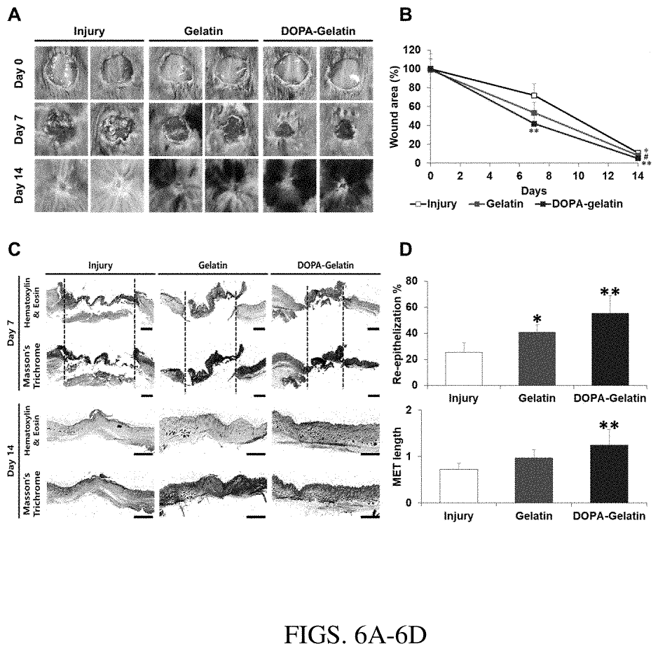

Full-thickness wounds on the dorsal skin of mice were made. Pure gelatin and DOPA-Gelatin were applied to the wounds and the gross morphology was assessed on day 0, 7, and 14 (FIG. 6A). On day 14, the injury group showed a central scab on the dorsum, while most wounds were healed in the DOPA-Gelatin group. In particular, hair regeneration was not observed around the wound in the injured group, but in all treatment groups (gelatin, DOPA-Gelatin), hair regeneration was found around the wound area. Interestingly, it was found that the DOPA-Gelatin group increased hair regeneration more than the other groups. Quantitative data of total wound contraction in FIG. 6B showed that at 7 days the injury group had the largest wound area of 71.79±12.25%. While among the treated groups, the DOPA-Gelatin group showed the fastest wound contraction rate with the wound area of 41.69±11.78%.

Histological Analysis

Histological study was also used to further examine the potency of DOPA-Gelatin on the skin wound healing process. The proliferation and migration of keratinocytes is a key feature of re-epithelialization during wound healing.^{[54]} Epithelialization with the same structure in vivo was accompanied by vasculogenesis, collagen deposition, and granularized tissue formation, which greatly promote tissue growth and healing. H&E staining (FIG. 6C) of day 7 post-injury demonstrates re-epithelialization was more pronounced in wounds treated with DOPA-Gelatin compared to open injury and gelatin groups. The re-epithelialization rate in the control, gelatin and DOPA-Gelatin groups were 25.6±7.2%, 41.0±5.9% and 55.4±13.7%, respectively (FIG. 6D). On the 14th day, every group had a healed wound while the DOPA-Gelatin group also increased hair growth. Masson trichrome staining of sections on 14th day post-injury showed enhanced collagen deposition in DOPA-Gelatin-treated mice, revealing a higher maturation level of collagen as compared to the control. This is suspected to be a result of greater fibroblast infiltration and proliferation in DOPA-Gelatin treated groups.

The histological study revealed faster wound healing in wound sites treated with DOPA-Gelatin in terms of increased wound contraction, enhanced collagen synthesis, more hair follicles, and higher re-epithelialization. These results support the ability of DOPA-Gelatin therapeutic material to be used as a skin-healing functional material.

### CONCLUSION

Mussel-inspired DOPA-Gelatin hydrogels are synthesized by a tyrosinase catalyzed one-step reaction. Desired mechanical strength and adhesion of DOPA-Gelatin lay the foundation of its application in skin wound healing. In vitro studies proved DOPA-Gelatin had desired biocompatibility and enhanced regenerative activities such as cell proliferation, migration, angiogenesis, and upregulation of wound healing related genes. In vivo experiments certified that DOPA-Gelatin can facilitate more rapid skin healing. The findings characterize the role of mussel-inspired DOPA-Gelatin in expediting the reparative process for skin wounds. The outstanding functional DOPA-Gelatin hydrogel should be further investigated and studied in other tissues as a mechanism of promoting tissue repair and regeneration.

### EXPERIMENTAL SECTION

Enzyme Mediated Synthesis of DOPA-Gelatin: 10% (w/w) gelatin solution (type A, G1890, Sigma, CA, USA) was prepared by dissolving 1 g gelatin in 10 g Milli-Q water at 80° C. for 1 h. The stock solution of tyrosinase (10 U/μL) was made by adding 50 kU tyrosinase powder (T3824, Sigma, MO, USA) into 5 mL Dulbecco's phosphate buffer saline (DPBS, pH6.5, Gibco, CA, USA) at room temperature. Tyrosinase stock solutions with volumes of 0, 5, 10, 20, and 50 μL were added into 1 mL gelatin solutions, respectively, to make the gelatin solutions with different tyrosinase concentration (0, 50, 100, 200, 500 U/mL). Corresponding samples were named as Tyr0, Tyr50, Tyr100, Tyr200, Tyr500. The reactions were performed at 37° C. in the Eppendorf ThermoMixer® C (Eppendorf, NY, USA) with an oscillating frequency of 2000 rpm. After the specified incubation time, the temperature was increased to 65° C. for one hour to inactivate the enzyme. The DOPA-Gelatin solutions were used immediately or stored at −80° C. for later use.

UV-Visible Spectroscopy: Gelatin solutions catalyzed by tyrosinase were monitored by a spectrophotometer DeNovix® DS11-FX (DeNovix, DE, USA). Two microliter reaction mixtures were collected and scanned at wavelengths from 220 nm to 500 nm. DOPA contents were analyzed at the wavelength of 280 nm.^{[21]}

FTIR Analysis: Tyr0 to Tyr500 samples and pure L-dopamine were characterized by Fourier transform infrared spectroscopy (JASCO, FT/IR-420, MD, USA) over the range of 400-4000 cm⁻¹ with 128 scans at 1 cm⁻¹ resolution. All samples were freeze-dried and ground with mortar and pestle into a fine powder. The potassium bromide (KBr) pellets were made with the sample weight content of 1%.

Quantification of DOPA Contents: Arnow's method was employed to determine the content of DOPA and its further oxidized derivatives.^{[31]} Three reagents were prepared to quantify the DOPA contents. Reagent A: 0.5 M HCl solution; reagent B: nitrite-molybdate solution (10 g NaNO₂ and 10 g Na₂MoO₄ dissolved in 100 mL water); and reagent C: 1 M NaOH solution (4 g sodium hydroxide dissolved in 100 mL water). Pure DOPA was used to make standard solutions with different concentrations of 0.02 mg/mL, 0.04 mg/mL, 0.06 mg/mL, 0.08 mg/mL and 0.1 mg/mL. 1 mL of water was used as a blank control. One milliliter of each standard solution, and each sample to be measured was placed in a tube and followed by adding 1 mL reagent A, followed by vortexing. Reagents B and C (1 mL each) were then added in rapid succession at room temperature, and each tube was mixed briefly on a vortexer. Each sample was assayed immediately on the spectrophotometer (DeNovix® DS11-FX) to characterize absorbance at 520 nm.

Lap Shear and Burst Pressure Tests: The samples were strained until failure in lap shear using an Instron® 5943 mechanical tester (MA, USA) equipped with a 100-N load cell with a cross-head speed of 1 mm/min. Samples of 20 μL were applied on a 10 mm×20 mm area of one glass slide, after which another glass slide was placed over this area and then placed at 4° C. for 1 h. Each sample was tested at least three times. To investigate burst pressure of the DOPA-modified gelatin, the sealing capability was measured according to a modified ASTM standard, F2392-04, for burst pressure, as previously described.^{[70,71]} Briefly, circular collagen sheets with a diameter of 30 mm were immersed in DPBS prior to sample preparation. A circular defect area with a diameter of 3 mm was punched in the center of the collagen sheet. 20 μL samples were pipette on the defect area and put at 4° C. for five minutes before testing. The collagen sheet with a sample on it was then fixed in the middle of two stainless steel annuli, using a custom-made burst pressure apparatus in which the upper annuli contained a 10-mm-diameter hole. Then, the air was applied to the system by a syringe (50 mL) pump at a speed of 20 mm/min. The maximum burst pressure was recorded by SPARKvue® (PASCO Scientific, CA, USA) software (n≥3).

Rheology Analysis: Rheological properties of DOPA-Gelatin hydrogels were evaluated by a rheometer (AR-G2, TA instruments protocol). Storage moduli, loss moduli and viscosity were measured with a parallel stainless metal plate geometry with a diameter of 25 mm. Before testing, all samples were equilibrated at 37° C. for 1 h. To prevent water evaporation, mineral oil was added around the plate after samples were loaded. The storage moduli, loss moduli, and viscosity were recorded by Anton Paar Rheocompass™ software.

Live/dead Assay: HDF cells and HaCaT cells were cultured in a humidified incubator (37° C., 5% CO2) using Dulbecco's Modified Eagle Medium (DMEM; Gibco, CA, USA) supplemented with 10% fetal bovine serum (FBS, Gibco, CA, USA) and 1% penicillin/streptomycin (Gibco, CA, USA). Cell viability was evaluated using a live/dead viability kit (LIVE/DEAD® Viability/Cytotoxicity Kit, Invitrogen, USA). Samples were coated on the bottom of the chambers. Cells (105 per chamber, passage four) were then seeded on the coated samples and incubated at 37° C. for 1, 3, and 7 days. The stained cells were imaged by a fluorescent microscope (Zeiss Axio Observer; Carl Zeiss, Jena, Germany). For each time point, samples were analyzed in triplicate. Image J software (NIH, MD, USA) was used to count the number of live and dead cells. Cell viability (%) was expressed as the ratio of living cells to total cell numbers in mean±SD. Proliferation (%) was calculated by the ratio of cell concentration on day 1, 3, 7 to the original seeding concentration in mean±SD.

Cell Migration Assay: Samples were coated evenly onto 150 mm diameter petri dishes and seeded at 106 HDF and HaCaT cells followed by incubation (37° C., 5% CO2). When the cells grew to full confluence on the dish, cells were scratched by a scratcher tip. After the scratch was made, the dish was gently washed to get rid of detached cells. Medium was then replenished with fresh medium without serum to suppress cell proliferation. Dishes were placed in the incubator for 0, 6, 12, and 24 hours. Cells were imaged using an inverted microscope (Zeiss Axio Observer; Carl Zeiss, Jena, Germany) prior to collection for RT-PCR gene expression analysis. The wound contraction was defined as Equation 1:

$$Wound contraction ( \% ) = ( \frac{{A}_{0} - {A}_{t}}{{A}_{0}} ) \times 100 \% \qquad Eq . ( 1 )$$

in which A₀ is the area of the wound measured immediately after scratching (0 h), and Aₜ is the area of the wound measured at time t (t=6, 12, 24 h) after the scratch. The wound area was calculated by manually tracing the cell-free area in images and counted by Image J software (NIH, MD, USA).

RT-PCR Assay: Total RNA was isolated from HDF and HaCaT cells using Qiazol lysis reagent (Qiagen, CA, USA) according to the manufacturer's instructions. One microgram of total RNA was transcribed into cDNA with a QuantiTect Reverse Transcription Kit (Qiagen). A Rotor-Gene SYBR Green PCR Kit (Qiagen) was used to perform real-time PCR (initial denaturation for 5 min at 95° C.; 45 cycles of denaturation for 5 s at 95° C. and amplification for 10 s at 60° C.).

Angiogenesis Assay: For the direct method, 250 μL of Matrigel (Corning Inc, NY, USA) was placed into each well (24-well plate). The plate was then incubated in a humid chamber for 30 min to allow for the formation of the gel structure. HUVECs (passage 4-6) were seeded (about 1.5×10⁴ cells/well). 100 μL of conditioned media (Promocell, Heidelberg, Germany) with 60 μL gelatin or DOPA-Gelatin was supplemented at each condition. The assay was run for 6 hours in a humidified chamber. The angiogenesis of HUVECs was imaged by an inverted fluorescence microscope (Zeiss Axio Observer; Carl Zeiss, Jena, Germany). For the indirect method, 250 μL Matrigel and 100 μL cell culture supernatant plus HUVEC cells were added into the well. Other conditions were the same as the direct method.

In Vivo Biodegradation and Wound Healing Studies: All animal experiments were approved by the UCLA Animal Research Committee. The animal experiments conducted aligned with relevant guidelines. Seven-week-old male mice, with body weight around 20 grams, were bought from Jackson Laboratory (Sacramento, CA) and fed and housed in clean cages maintained at 25° C. At the start of the experiments, mice were anesthetized by inhalation of isoflurane (1.5% in 100% O₂). Anesthesia was maintained throughout the survival surgery. Dorsal skin was shaved and cleaned with an iodophor (0.2% w/v). The dorsal skin was then surgically excised to create a full-thickness circular skin defect area (diameter around 1 cm). Three groups, including no-treatment (injury), pure gelatin (Gelatin), and Tyr100 DOPA-modified gelatin (DOPA-Gelatin), were prepared. Each wound of the treatment group was evenly covered with 200 μL of the corresponding samples. Wound healing was evaluated by measuring the wound area size by a digital caliper and capturing pictures on certain days (day 0, 7, and 14). The wound area was calculated according to Equation 2:

$$Wound area ( \% ) = ( \frac{{A}_{0} - {A}_{t}}{{A}_{0}} ) \times 100 \% \qquad Eq . ( 2 )$$

where A₀ and Aₜ are the wound area on day 0 and wound area on day t, respectively. For degradation tests, pure gelatin and DOPA-Gelatin labeled with fluorescein isothiocyanate (FTIC, Sigma) were applied to the wound site for 0, 7, and 14 days and fluorescent images were taken by fluorescent microscope (Zeiss Axio Observer; Carl Zeiss, Jena, Germany).

Histological Analysis: Mice were sacrificed using CO2 on specified days (0, 7, and 14 days). Sample applied to skin tissue was immediately collected and fixed in 10% neutral buffered formalin (Leica Biosystems, IL, USA). Fixed tissues were processed using standard methods and embedded in paraffin blocks. The blocks were sectioned to 4 μm in thickness, and the sections were stained by hematoxylin and eosin (H&E) stain and Masson trichrome (MT) stain. Histology images were acquired on a Nikon inverted microscope.

Statistical Analysis: Data are displayed as mean±standard deviation (SD). All statistical analyses and graphs were carried out by SPSS Statistics software (IBM, IL, USA) and GraphPad Prism 8.0 (GraphPad Software, CA, USA). Multiple comparisons were analyzed using one-way ANOVA with Tukey post hoc tests for more than a triplicate of group data sets. P<0.05 is considered as significant.

While embodiments of the present invention have been shown and described, various modifications may be made without departing from the scope of the present invention. The invention, therefore, should not be limited, except to the following claims, and their equivalents.

### REFERENCES

[1] S. Dhivya, V. V. Padma, E. Santhini, Biomedicine (Taipei) 2015, 5, 22.
[2] C. Yu, Z.-Q. Hu, R.-Y. Peng, Military Medical Research 2014, 1, 24.
[3] X. Sun, H. Zhang, J. He, R. Cheng, Y. Cao, K. Che, L. Cheng, L. Zhang, G. Pan, P. Ni, L. Deng, Y. Zhang, H. C. l. A. Santos, W. Cui, Applied Materials Today 2018, 13, 54.
[4] Y. Zhou, L. Gao, J. Peng, M. Xing, Y. Han, X. Wang, Y. Xu, J. Chang, Advanced Healthcare Materials 2018, 7, 1800144.
[5] H. Ma, Q. Zhou, J. Chang, C. Wu, ACS Nano 2019, 13, 4302.
[6] S. S. S. Wang, P.-L. Hsieh, P.-S. Chen, Y.-T. Chen, J.-S. Jan, Colloids and Surfaces B: Biointerfaces 2013, 111, 423.
[7] W. J. Park, R. S. Hwang, I.-S. Yoon, Molecules 2017, 22, 1259.
[8] Y. Dong, W. U. Hassan, R. Kennedy, U. Greiser, A. Pandit, Y. Garcia, W. Wang, Acta Biomaterialia 2014, 10, 2076.
[9] J. Koehler, F. P. Brandl, A. M. Goepferich, European Polymer Journal 2018, 100, 1
[10] T. Chen, Y. Chen, H. U. Rehman, Z. Chen, Z. Yang, M. Wang, H. Li, H. Liu, ACS Applied Materials & Interfaces 2018, 10, 33523.
[11] B. P. Lee, P. B. Messersmith, J. N. Israelachvili, J. H. Waite, Annual Review of Materials Research 2011, 41, 99.
[12] D. S. Hwang, H. Zeng, Q. Lu, J. Israelachvili, J. H. Waite, Soft Matter 2012, 8, 5640.
[13] J. Xu, G. M. Soliman, J. Barralet, M. Cerruti, Langmuir 2012, 28, 14010.
[14] Y. Liu, H. Meng, S. Konst, R. Sarmiento, R. Rajachar, B. P. Lee, ACS Applied Materials & Interfaces 2014, 6, 16982.
[15] J. Shin, J. S. Lee, C. Lee, H.-J. Park, K. Yang, Y. Jin, J. H. Ryu, K. S. Hong, S.-H. Moon, H.-M. Chung, H. S. Yang, S. H. Um, J.-W. Oh, D.-I. Kim, H. Lee, S.-W. Cho, Advanced Functional Materials 2015, 25, 3814.
[16] B. Gao, L. Chen, Y. Zhao, X. Yan, X. Wang, C. Zhou, Y. Shi, W. Xue, European Polymer Journal 2019, 110, 192.
[17] H. Lee, S. M. Dellatore, W. M. Miller, P. B. Messersmith, Science 2007, 318, 426.
[18] L. A. Burzio, V. A. Burzio, J. Pardo, L. O. Burzio, Comparative Biochemistry and Physiology Part B: Biochemistry and Molecular Biology 2000, 126, 383.
[19] S.-Y. Seo, V. K. Sharma, N. Sharma, Journal of Agricultural and Food Chemistry 2003, 51, 2837.
[20] K. Min, D.-H. Park, Y. J. Yoo, Journal of Biotechnology 2010, 146, 40.
[21] Y. Chan Choi, J. S. Choi, Y. J. Jung, Y. W. Cho, Journal of Materials Chemistry B 2014, 2, 201.
[22] C. M. Elvin, T. Vuocolo, A. G. Brownlee, L. Sando, M. G. Huson, N. E. Liyou, P. R. Stockwell, R. E. Lyons, M. Kim, G. A. Edwards, G. Johnson, G. A. McFarland, J. A. M. Ramshaw, J. A. Werkmeister, Biomaterials 2010, 31, 8323.
[23] G. A. Gonzalez-Aguilar, A. A. Kader, J. K. Brecht, P. M. A. Toivonen, in Postharvest Biology and Technology of Tropical and Subtropical Fruits, DOI: https://doi.org/10.1533/9780857093622.381 (Ed: E. M. Yahia), Woodhead Publishing 2011, p. 381.
[24] F. V. Marques Silva, A. Sulaiman, in Encyclopedia of Food Chemistry, DOI: https://doi.org/10.1016/B978-0-08-100596-5.21636-3 (Eds: L. Melton, F. Shahidi, P. Varelis), Academic Press, Oxford 2019, p. 287.
[25] X. Yang, L. Zhu, S. Tada, D. Zhou, T. Kitajima, T. Isoshima, Y. Yoshida, M. Nakamura, W. Yan, Y. Ito, Int J Nanomedicine 2014, 9, 2753.
[26] M. Mehdizadeh, H. Weng, D. Gyawali, L. Tang, J. Yang, Biomaterials 2012, 33, 7972.
[27] N. Tajima, M. Takai, K. Ishihara, Analytical Chemistry 2011, 83, 1969.
[28] C. K. Song, M.-K. Kim, J. Lee, E. Davaa, R. Baskaran, S.-G. Yang, Macromolecular Research 2019, 27, 119.
[29] M. Weinhold, S. Soubatch, R. Temirov, M. Rohlfing, B. Jastorff, F. S. Tautz, C. Doose, The Journal of Physical Chemistry B 2006, 110, 23756.
[30] A. Ledeti, G. Vlase, D. Circioban, I. Ledeti, L. Stelea, T. Vlase, A. Caunii, Rev Chim 2016, 67, 2648.
[31] L. E. Arnow, J biol. Chem 1937, 118, 531.
[32] J. H. Waite, M. L. Tanzer, Analytical Biochemistry 1981, 111, 131.
[33] R. Hafidz, C. Yaakob, I. Amin, A. Noorfaizan, International Food Research Journal 2011, 18, 813.
[34] B. Duncan, in Advances in Structural Adhesive Bonding, DOI: https://doi.org/10.1533/9781845698058.3.389 (Ed: D. A. Dillard), Woodhead Publishing 2010, p. 389.
[35] D. J. Dos Santos, D. J. Carastan, L. B. Tavares, G. F. Batalha, in Comprehensive Materials Processing, DOI: https://doi.org/10.1016/B978-0-08-096532-1.00205-3 (Eds: S. Hashmi, G. F. Batalha, C. J. Van Tyne, B. Yilbas), Elsevier, Oxford 2014, p. 37.
[36] C. Mahendra Kumar, U. V. Sathisha, S. Dharmesh, A. G. A. Rao, S. A. Singh, Biochimie 2011, 93, 562.
[37] J. Yang, M. A. Cohen Stuart, M. Kamperman, Chemical Society Reviews 2014, 43, 8271.
[38] A. H. Hofman, I. A. van Hees, J. Yang, M. Kamperman, Advanced Materials 2018, 30, 1704640.
[39] R. Mirshafian, W. Wei, J. N. Israelachvili, J. H. Waite, Biochemistry 2016, 55, 743.
[40] M. Yu, J. Hwang, T. J. Deming, Journal of the American Chemical Society 1999, 121, 5825.
[41] Y. Hong, F. Zhou, Y. Hua, X. Zhang, C. Ni, D. Pan, Y. Zhang, D. Jiang, L. Yang, Q. Lin, Y. Zou, D. Yu, D. E. Arnot, X. Zou, L. Zhu, S. Zhang, H. Ouyang, Nature Communications 2019, 10, 2060.
[42] O. Pinkas, D. Goder, R. Noyvirt, S. Peleg, M. Kahlon, M. Zilberman, Acta Biomaterialia 2017, 51, 125.
[43] F. Scognamiglio, A. Travan, I. Rustighi, P. Tarchi, S. Palmisano, E. Marsich, M. Borgogna, I. Donati, N. de Manzini, S. Paoletti, Journal of Biomedical Materials Research Part B: Applied Biomaterials 2016, 104, 626.
[44] N. Annabi, Y.-N. Zhang, A. Assmann, E. S. Sani, G. Cheng, A. D. Lassaletta, A. Vegh, B. Dehghani, G. U. Ruiz-Esparza, X. Wang, S. Gangadharan, A. S. Weiss, A. Khademhosseini, Sci Transl Med 2017, 9, eaai7466.
[45] J. M. Zuidema, C. J. Rivet, R. J. Gilbert, F. A. Morrison, Journal of Biomedical Materials Research Part B: Applied Biomaterials 2014, 102, 1063.
[46] H. Wang, Y. Wu, C. Cui, J. Yang, W. Liu, Adv Sci (Weinh) 2018, 5, 1800711.
[47] A. Lejardi, R. Hernandez, M. Criado, J. I. Santos, A. Etxeberria, J. R. Sarasua, C. Mijangos, Carbohydrate Polymers 2014, 103, 267.
[48] K. Muniandy, S. Gothai, W. S. Tan, S. S. Kumar, N. Mohd Esa, G. Chandramohan, K. S. Al-Numair, P. Arulselvan, Evid Based Complement Alternat Med 2018, 2018, 3142073.
[49] T. J. Shaw, P. Martin, Journal of Cell Science 2009, 122, 3209.
[50] S. Werner, T. Krieg, H. Smola, Journal of Investigative Dermatology 2007, 127, 998.
[51] C.-W. Wong, C. F. LeGrand, B. F. Kinnear, R. M. Sobota, R. Ramalingam, D. E. Dye, M. Raghunath, E. B. Lane, D. R. Coombe, Scientific Reports 2019, 9, 18561.
[52] Y. Shi, T. L. Xing, H. B. Zhang, R. X. Yin, S. M. Yang, J. Wei, W. J. Zhang, Biomedical Materials 2018, 13, 035008.
[53] W.-G. La, S. H. Bhang, J.-Y. Shin, H. H. Yoon, J. Park, H. S. Yang, S.-H. Yu, Y.-E. Sung, B.-S. Kim, Biotechnology Progress 2012, 28, 1055.
[54] S. Babitha, P. S. Korrapati, Biomedical Materials 2017, 12, 055008.
[55] A. D. Metcalfe, M. W. J. Ferguson, Journal of The Royal Society Interface 2007, 4, 413.
[56] L. Germain, A. Jean, F. o. A. Auger, D. R. Garrel, Journal of Surgical Research 1994, 57, 268.
[57] G. Serini, M.-L. Bochaton-Piallat, P. Ropraz, A. Geinoz, L. Borsi, L. Zardi, G. Gabbiani, The Journal of cell biology 1998, 142, 873.
[58] R. F. Diegelmann, M. C. Evans, Frontiers in Bioscience 2004, 9, 283.
[59] P. Bao, A. Kodra, M. Tomic-Canic, M. S. Golinko, H. P. Ehrlich, H. Brem, J Surg Res 2009, 153, 347.
[60] G. Schultz, W. Clark, D. S. Rotatori, Journal of Cellular Biochemistry 1991, 45, 346.
[61] N. X. Landén, D. Li, M. Ståhle, Cellular and Molecular Life Sciences 2016, 73, 3861.
[62] F. Cheng, Y. Shen, P. Mohanasundaram, M. Lindström, J. Ivaska, T. Ny, J. E. Eriksson, Proceedings of the National Academy of Sciences 2016, 113, E4320.
[63] S. E. Gill, W. C. Parks, Int J Biochem Cell Biol 2008, 40, 1334.
[64] B. Mulholland, S. J. Tuft, P. T. Khaw, Eye 2005, 19, 584.
[65] N. Ojeh, I. Pastar, M. Tomic-Canic, O. Stojadinovic, International Journal of Molecular Sciences 2015, 16.
[66] T. Velnar, T. Bailey, V. Smrkolj, Journal of International Medical Research 2009, 37, 1528.
[67] S. A. Eming, P. Martin, M. Tomic-Canic, Sci Transl Med 2014, 6, 265sr6.
[68] Z. Yang, Q. Tu, Y. Zhu, R. Luo, X. Li, Y. Xie, M. F. Maitz, J. Wang, N. Huang, Advanced Healthcare Materials 2012, 1, 548.
[69] T. Li, H. Ma, H. Ma, Z. Ma, L. Qiang, Z. Yang, X. Yang, X. Zhou, K. Dai, J. Wang, ACS Applied Materials & Interfaces 2019, 11, 17134.
[70] A. Assmann, A. Vegh, M. Ghasemi-Rad, S. Bagherifard, G. Cheng, E. S. Sani, G. U. Ruiz-Esparza, I. Noshadi, A. D. Lassaletta, S. Gangadharan, A. Tamayol, A. Khademhosseini, N. Annabi, Biomaterials 2017, 140, 115.
[71] E. Shirzaei Sani, A. Kheirkhah, D. Rana, Z. Sun, W. Foulsham, A. Sheikhi, A. Khademhosseini, R. Dana, N. Annabi, Science Advances 2019, 5, eaav1281.

All publications mentioned herein (e.g., the references numerically listed above) are incorporated herein by reference to disclose and describe aspects, methods and/or materials in connection with the cited publications.

1. A method of making 3, 4-dihydroxyphenylalanine (DOPA)-Gelatin comprising:
providing a solution containing porcine gelatin;
incubating the solution containing porcine gelatin with tyrosinase; such that 3, 4-dihydroxyphenylalanine (DOPA)-is made.

2. The method of making (DOPA)-Gelatin of claim 1, wherein the solution containing porcine gelatin is incubated with tyrosinase to form 3, 4-dihydroxyphenylalanine (DOPA)-Gelatin selected so that:
the 3, 4-dihydroxyphenylalanine (DOPA)-Gelatin exhibits a shear strength of at least 2 MPa;
the 3, 4-dihydroxyphenylalanine (DOPA)-Gelatin exhibits a burst pressure of at least 6 kPa;
the 3, 4-dihydroxyphenylalanine (DOPA)-Gelatin exhibits a load force of at least 60 N; and
the 3, 4-dihydroxyphenylalanine (DOPA)-Gelatin exhibits a tensile stress of at least 3 MPa.

3. The method of making (DOPA)-Gelatin of claim 2, wherein the solution containing porcine gelatin is incubated with tyrosinase for more than 30 minutes or more than 60 minutes.

4. The method of making (DOPA)-Gelatin of claim 1, wherein the concentration of tyrosinase is between 100-200 U/mL.

5. The method of making (DOPA)-Gelatin of claim 1, further comprising heating the solution to inactivate the tyrosinase.

6. The method of making (DOPA)-Gelatin of claim 1, wherein the 3, 4-dihydroxyphenylalanine (DOPA)-Gelatin is made in a one-step synthesis reaction.

7. A 3, 4-dihydroxyphenylalanine (DOPA)-Gelatin composition made by the method of claim 1.

8. A therapeutic composition of matter comprising porcine gelatin having substantially all of the tyrosine residues converted to 3, 4-dihydroxyphenylalanine (DOPA).

9. The therapeutic composition of matter of claim 8, wherein the composition is substantially free of metallic ions.

10. The therapeutic composition of matter of claim 8, wherein the composition is sterile and comprises a pharmaceutically acceptable carrier.

11. The therapeutic composition of claim 8, wherein the 3, 4-dihydroxyphenylalanine (DOPA)-Gelatin exhibits a shear strength of at least 2 MPa.

12. The therapeutic composition of claim 8, wherein the 3, 4-dihydroxyphenylalanine (DOPA)-Gelatin exhibits a burst pressure of at least 6 kPa.

13. The therapeutic composition of claim 8, wherein the 3, 4-dihydroxyphenylalanine (DOPA)-Gelatin exhibits a load force of at least 60 N.

14. The therapeutic composition of claim 8, wherein the 3, 4-dihydroxyphenylalanine (DOPA)-Gelatin exhibits a tensile stress of at least 3 MPa.

15. The therapeutic composition of claim 8, wherein the 3, 4-dihydroxyphenylalanine (DOPA)-Gelatin exhibits a storage modulus of at least 700 Pa.

16. The therapeutic composition of claim 8, further comprising at least one additional therapeutic agent selected from: an antibiotic, an anti-inflammatory agent, a hemostatic agent, an embolic agent, and a chemotherapeutic agent.

17. The therapeutic composition of claim 8, wherein:
at least 90% of the tyrosine residues of the porcine gelatin have been converted to 3, 4-dihydroxyphenylalanine (DOPA);
the composition is sterile and comprises a pharmaceutically acceptable carrier;
the 3, 4-dihydroxyphenylalanine (DOPA)-Gelatin exhibits a shear strength of at least 2 MPa;
the 3, 4-dihydroxyphenylalanine (DOPA)-Gelatin exhibits a burst pressure of at least 6 kPa;
the 3, 4-dihydroxyphenylalanine (DOPA)-Gelatin exhibits a load force of at least 60 N; and
the 3, 4-dihydroxyphenylalanine (DOPA)-Gelatin exhibits a tensile stress of at least 3 MPa.

18. A method of delivering a composition of claim 8 to a preselected site comprising:
disposing the composition in a vessel having a first end comprising an opening and a second end;
applying a force to the second end of the vessel, wherein the force is sufficient to move the composition out of the vessel through the opening; and
delivering the composition out of the vessel through the opening and to the preselected site.

19. The method of claim 18, wherein the site is an in vivo site.

20. The method of claim 1, wherein the site is at an in vivo location where an individual has experienced trauma or injury.
